(19) United States
(12) Patent Application Publication
(10) Pub. No.: US 2017/0010458 A1
(43) Pub. Date: Jan. 12, 2017

(54) ENDOSCOPE

(71) Applicant: OLYMPUS CORPORATION, Tokyo (JP)

(72) Inventor: Yoshikazu NISHIJIMA, Uenohara-shi (JP)

(73) Assignee: OLYMPUS CORPORATION, Tokyo (JP)

(21) Appl. No.: 15/274,042

(22) Filed: Sep. 23, 2016

Related U.S. Application Data
(63) Continuation of application No. PCT/JP2014/067698, filed on Jul. 2, 2014.

(30) Foreign Application Priority Data
Mar. 25, 2014 (JP) 2014-062214

(51) Int. Cl. G02B 23/24; A61B 1/005; F16L 15/00; A61B 1/00

(52) U.S. Cl. CPC G02B 23/2476; A61B 1/00105; A61B 1/0055; F16L 15/006

(57) ABSTRACT

An endoscope includes a bending portion, a flexible tube portion having a tubular flexible member, and a connecting member having a cylindrical shape, the connecting member being configured to connect a proximal end portion of the bending portion and a distal end portion of the flexible tube portion, and a distal end thin wall portion of the flexible member such as a flex in the flexible tube portion is held in a sandwiched manner by a double tube portion which forms a gap formed at a proximal end portion of the connecting member and having the cylindrical shape.

### CROSS REFERENCE TO RELATED APPLICATION

This application is a continuation application of PCT/JP2014/067698 filed on Jul. 2, 2014 and claims benefit of Japanese Application No. 2014-062214 filed in Japan on Mar. 25, 2014, the entire contents of which are incorporated herein by this reference.

### BACKGROUND OF THE INVENTION

1. Field of the Invention

The present invention relates to an endoscope, and more particularly to an endoscope including an insertion portion which has a flexible tube portion and a bending portion.

2. Description of the Related Art

Conventionally, endoscopes have been widely used in industrial fields and medical fields. Some endoscopes include an elongated and flexible insertion portion. The insertion portion includes a bending portion on the distal end side thereof, and a flexible tube portion connected to the proximal end side of the bending portion. The bending portion is configured to be bendable by bending operation performed at an operation portion of the endoscope. The flexible tube portion includes a tubular flexible member in which various kinds of internal components are inserted. The bending portion and the flexible tube portion are connected to each other with a connection pipe sleeve, and the insertion portion including the connecting part with the connection pipe sleeve is covered with resin or the like.

The connection pipe sleeve as a connecting member is fixed so as to closely contact the inner circumferential surface of the distal end side of the flexible member of the flexible tube portion, or so as to closely contact the outer circumferential surface of the distal end side of the flexible member in the flexible portion. For example, Japanese Patent Application Laid-Open Publication No. 2-46819 discloses a configuration in which the connection pipe sleeve is internally fitted to the flexible tube portion from the opening on the distal end side of the spiral tube of the flexible tube portion, to be fitted to the inner circumferential surface of the flexible tube portion. In addition, Japanese Patent Application Laid-Open Publication No. 2007-298815 discloses a configuration in which the connection pipe sleeve is externally fitted to the flexible tube portion from the opening on the distal end side of the flexible tube portion, to be fitted to the outer circumferential surface of the flexible tube portion.

### SUMMARY OF THE INVENTION

According to one aspect of the present invention, it is possible to provide an endoscope including: a bending portion; a flexible tube portion including a tubular flexible member; a connecting member having a cylindrical shape, the connecting member being configured to connect a proximal end portion of the bending portion and a distal end portion of the flexible tube portion; and a holding portion configured to hold a thin wall portion of the flexible member in a sandwiching manner by a double tube portion which forms a gap formed at a proximal end portion of the connecting member and having the cylindrical shape.

### BRIEF DESCRIPTION OF THE DRAWINGS

FIG. 1 is a configuration diagram showing a configuration of an endoscope apparatus according to a first embodiment of the present invention.

FIG. 2 is a cross-sectional view of a connecting part of a bending portion 8 and a flexible tube portion 9 according to the first embodiment of the present invention.

FIG. 3 is a cross-sectional view of a connecting member 11 before a proximal end side part 13L of a cylindrical member 13 is brought into close contact with an outer circumferential portion of the flexible tube portion 9, according to the first embodiment of the present invention.

FIG. 4 is a cross-sectional view showing a state where a distal end thin wall portion 9a of the flexible tube portion 9 is inserted in a gap g of the connecting member 11 before the proximal end side part 13L of the cylindrical member 13 is brought into close contact with the outer circumferential portion of the flexible tube portion 9, according to the first embodiment of the present invention.

FIG. 5 illustrates a method of bringing the proximal end side part 13L of the cylindrical member 13 into close contact with the outer circumferential portion of the distal end thin wall portion 9a of the flexible tube portion 9 by performing swaging processing on the connecting member 11, according to the first embodiment of the present invention.

FIG. 6 illustrates a state before the distal end thin wall portion 9a of the flexible tube portion 9 is externally fitted to a proximal end side part 12L of a cylindrical member 12A and a cylindrical member 13B is put over the distal end thin wall portion 9a of the flexible tube portion 9, according to a modified example 2 of the first embodiment of the present invention.

FIG. 7 illustrates a state after the swaging processing has been performed after externally fitting the distal end thin wall portion 9a of the flexible tube portion 9 to a proximal end side part 12L of a cylindrical member 12A and then putting a cylindrical member 13B over the distal end thin wall portion 9a of the flexible tube portion 9, according to the modified example 2 of the first embodiment of the present invention.

FIG. 8 is a cross-sectional view of the connecting member 11 according to a modified example 3 of the first embodiment of the present invention.

FIG. 9 is a cross-sectional view of a connecting member 11A according to a second embodiment of the present invention.

### DETAILED DESCRIPTION OF THE PREFERRED EMBODIMENTS

Hereinafter, embodiments of the present invention will be described with reference to drawings.

Note that, in the drawings used in the description below, a different scale size is used for each of the constituent elements in order to allow each of the constituent elements to be illustrated in a recognizable size in the drawings, and the present invention is not limited only to the number, shapes, ratio of the sizes of the constituent elements, and a relative positional relationship among the constituent elements shown in these drawings.

### First Embodiment

### Configuration of Endoscope Apparatus

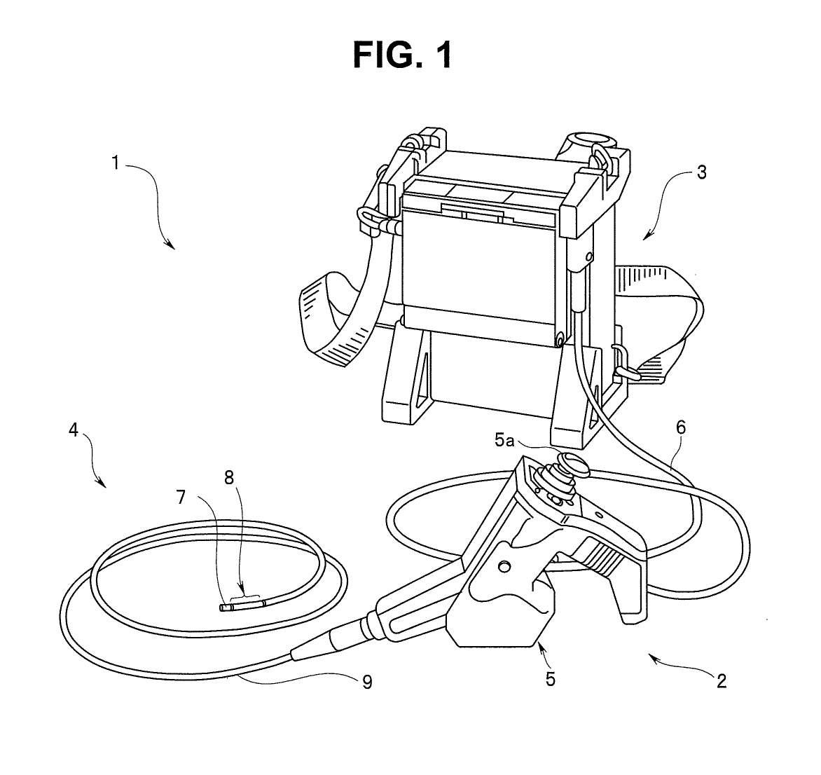

FIG. 1 is a configuration diagram showing a configuration of an endoscope apparatus according to the present embodiment of the present invention.

As shown in FIG. 1, an endoscope system 1 includes an endoscope 2, and an apparatus body 3 connected to the endoscope 2.

The endoscope 2 is configured by including an elongated flexible insertion portion 4, an operation portion 5 connected to the proximal end portion of the insertion portion 4, and a universal cord 6 extended from the operation portion 5.

The insertion portion 4 includes continuously in the following order from the distal end side thereof, a distal end rigid portion 7, a bending portion 8, and a long flexible tube portion 9. The operation portion 5 is connected to the proximal end portion of the flexible tube portion 9.

The bending portion 8 can be bent in four directions, that is, up, down, left and right directions, for example, by operating a joystick 5a provided at the operation portion 5. Note that the operation portion 5 is provided with not only the joystick 5a but also various kinds of switches, etc., for giving an instruction for photographing operation to be performed by an image pickup device (not shown) provided in the distal end rigid portion 7.

The apparatus body 3 is box-shaped, for example, and includes on an external housing thereof a monitor for displaying an endoscopic image picked up by the endoscope 2. (Connection configuration of flexible tube portion and bending portion)

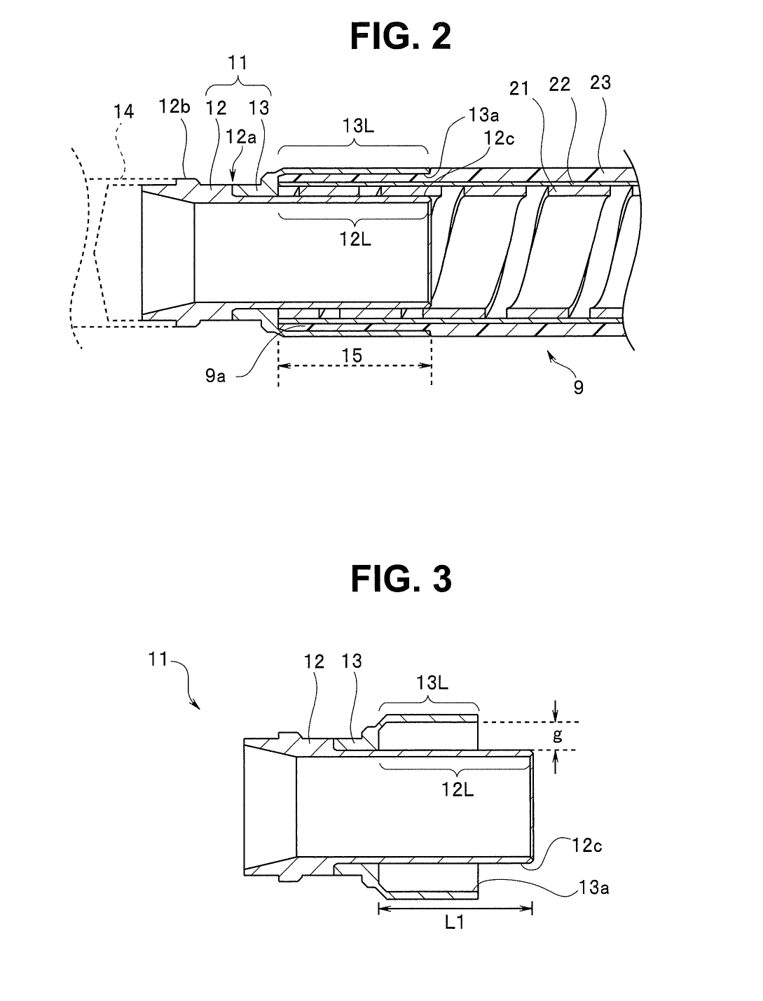

FIG. 2 is a cross-sectional view of a connecting part of the bending portion 8 and the flexible tube portion 9 according to the present embodiment. The bending portion 8 and the flexible tube portion 9 are connected to each other with a connecting member 11. The connecting member 11 is a cylindrical-shaped pipe sleeve made of metal such as stainless steel. The connecting member 11 is configured by two cylindrical members 12 and 13. A step portion 12a is formed on the outer circumferential side of the cylindrical member 12, and the distal end portion of the cylindrical member 13 is externally fitted to the cylindrical member 12 from the proximal end portion of the cylindrical member 12 such that the distal end portion of the cylindrical member 13 abuts the step portion 12a. The cylindrical member 13 is connected and fixed to the cylindrical member 12 by laser welding applied to the step portion 12a. Note that the cylindrical members 12 and 13 may be fixed to each other by adhesive.

The cylindrical member 12 includes a circumferential projection portion 12b on the outer circumferential portion on the distal end side with respect to the step portion 12a. The cylindrical member 12 is connected to the bending piece 14 such that the distal end portion of the cylindrical member 12 is internally fitted from the proximal end portion of the proximal-most bending piece 14 (shown by the dotted line) and the outer circumferential surface of the distal end portion of the cylindrical member 12 is fitted on the inner circumferential surface of the bending piece 14. The bending piece 14 (shown by the dotted line) and the cylindrical member 12 are fixed to each other by adhesive or welding, with the proximal end portion of the bending piece 14 (shown by dotted line) abutting the projection portion 12b.

The proximal end parts of the cylindrical members 12 and 13 have a shape in which a gap g is formed between an outer circumferential surface 12c of the proximal end part of the cylindrical member 12 and an inner circumferential surface 13a of the proximal end part of the cylindrical member 13, when the distal end part of the cylindrical member 13 is externally fitted to the cylindrical member 12 from the proximal end side of the cylindrical member 12, and the cylindrical member 12 and the cylindrical member 13 are connected and fixed to each other.

Specifically, an inner diameter of a proximal end side part 13L of the cylindrical member 13 is larger than an outer diameter of a proximal end side part 12L of the cylindrical member 12. The gap g is formed by the outer circumferential surface 12c of the proximal end side part 12L which is a part from the center part to the proximal end part of the cylindrical member 12 and the inner circumferential surface 13a of the proximal end side part 13L which is a part from the center part to the proximal end part of the cylindrical member 13.

A distal end thin wall portion 9a of the flexible tube portion 9 is arranged in the gap g in the state where the distal end thin wall portion 9a is pressed and sandwiched by the outer circumferential surface 12c of the proximal end side part 12L of the cylindrical member 12 and the inner circumferential surface 13a of the proximal end side part 13L of the cylindrical member 13.

The flexible tube portion 9 is configured by including a flex 21, a braid 22, and an outer sheath resin 23 which are layered in this order from the inner side. The flex 21 is a spiral tube which is a flexible member having a shape formed by winding a flat plate member in a spiral shape. The braid 22 is a metal braid. The outer sheath resin 23 is formed on the outer circumferential portion of the braid 22 such that a part of the outer sheath resin enters the fine clearances between the respective metal wires of the braid 22.

As described above, the flexible tube portion 9 includes a cylindrical flexible member, and the flexible member includes the flex 21, the outer sheath resin 23 that covers the flex 21, and the braid 22 provided between the flex 21 and the outer sheath resin 23.

In the configuration shown in FIG. 2, the proximal end side parts 12L and 13L that sandwich the distal end part of the flexible tube portion 9 have almost the same length.

As described later, the proximal end side part 13L of the cylindrical member 13 is pressed from the outer circumferential side toward the inner circumferential side by the swaging processing, thereby causing the proximal end side part 13L of the cylindrical member 13 and the proximal end side part 12L of the cylindrical member 12 to strongly hold the distal end thin wall portion 9a of the flexible tube portion 9 in a sandwiching manner from both sides. The swaging processing causes the distal end portion of the outer sheath resin 23 to be compressed to the extent that the thickness thereof is reduced. Therefore, the distal end thin wall portion 9a of the flexible tube portion 9 is sandwiched and firmly fixed between the outer circumferential surface 12c of the proximal end side part 12L of the cylindrical member 12 and the inner circumferential surface 13a of the proximal end side part 13L of the cylindrical member 13.

In other words, the distal end thin wall portion 9a of the flexible tube portion 9 is held by the outer circumferential surface 12c of the proximal end side part 12L of the cylindrical member 12 and the inner circumferential surface 13a of the proximal end side part 13L of the cylindrical member 13 over a predetermined distance L1 with a predetermined pressure.

As described above, the connecting member 11 is a pipe sleeve that connects the proximal end portion of the bending portion 8 and the distal end portion of the flexible tube portion 9. The proximal end side part 12L of the cylindrical member 12 and the proximal end side part 13L of the cylindrical member 13 that covers the proximal end side part 12L configure a double tube portion, and the double tube portion, which forms the cylindrical-shaped gap g formed at the proximal end portion of the connecting member 11, configures a holding portion that holds the distal end thin wall portion 9a which is a thin wall portion of the flexible member in a sandwiching manner.

Before the distal end thin wall portion 9a of the flexible tube portion 9 is inserted in the gap g, adhesive (not shown) is injected into the gap g. The distal end thin wall portion 9a of the flexible tube portion 9 is held in the sandwiched manner over the predetermined distance L1 with the predetermined pressure and also fixed with adhesive, which improves watertightness between the outer sheath resin 23 and the connecting member 11.

Note that, though not shown in FIG. 2, the insertion portion 4 is configured such that a braid or an outer sheath resin is further provided from the distal end side to the proximal end side, so as to cover the outer circumferential portions of the bending piece 14, the connecting member 11 and the outer sheath resin 23.

Next, description will be made on a method of pressing the outer sheath resin 23 of the flexible tube portion 9 from the outer circumferential side toward the inner circumferential side, to bring the proximal end side part 13L of the cylindrical member 13 into close contact with the outer circumferential portion of the flexible tube portion 9.

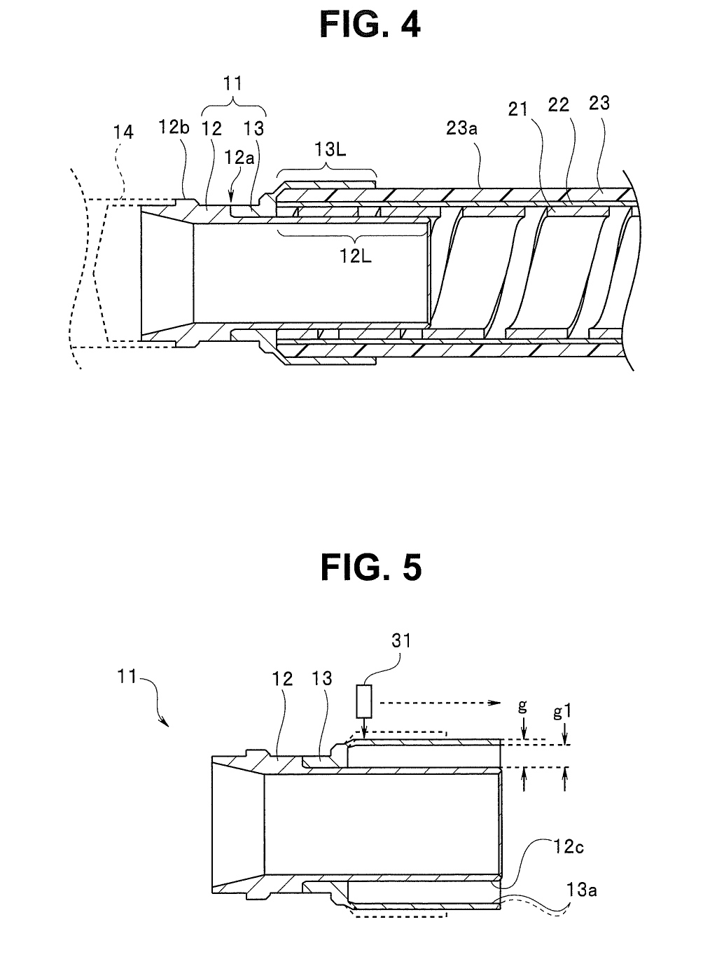

FIG. 3 is a cross-sectional view of the connecting member 11 before the proximal end side part 13L of the cylindrical member 13 is brought into close contact with the outer circumferential portion of the flexible tube portion 9. FIG. 4 is a cross-sectional view showing the state where the distal end thin wall portion 9a of the flexible tube portion 9 is inserted in the gap g of the connecting member 11 before the proximal end side part 13L of the cylindrical member 13 is brought into close contact with the outer circumferential portion of the flexible tube portion 9. FIG. 5 illustrates a method of bringing the proximal end side part 13L of the cylindrical member 13 into close contact with the outer circumferential portion of the distal end thin wall portion 9a of the flexible tube portion 9 by performing swaging processing on the connecting member 11.

As shown in FIG. 3, when the cylindrical members 12 and 13 are connected to each other, the gap g has a width that allows the distal end thin wall portion 9a of the flexible tube portion 9 to enter.

In addition, the proximal end side part 13L of the outer cylindrical member 13 is extended in the axial direction by the swaging processing. Therefore, the length in the axial direction of the proximal end side part 13L is set to be shorter than the length of the proximal end side part 12L of the inner cylindrical member 12. The proximal end side part 12L has the length enough for the proximal end side part 12L of the inner cylindrical member 12 to receive a force applied to the flexible tube portion 9 at the time of swaging processing of the proximal end side part 13L of the outer cylindrical member 13. Conversely, the proximal end side part 12L of the cylindrical member 12 has only to have a length set such that the length of the proximal end side part 13L becomes equal to or shorter than the length of the proximal end side part 12L when the swaging processing is finished.

The distal end thin wall portion 9a of the flexible tube portion 9 is inserted into the gap g. FIG. 4 shows the state where the distal end thin wall portion 9a of the flexible tube portion 9 is inserted in the gap g of the connecting member 11.

The connecting member 11 is placed on a swaging apparatus in the state where the distal end portion of the flexible tube portion 9 is inserted in the gap g between the outer circumferential surface 12c of the proximal end side part 12L of the cylindrical member 12 and the inner circumferential surface 13a of the proximal end side part 13L of the cylindrical member 13, and then swaging processing is performed on the connecting member. Before performing the swaging processing, adhesive (not shown) is poured in the gap g. Note that the adhesive has only to be provided at least between the outer circumferential surface 23a of the outer sheath resin 23 and the outer circumferential surface 12c of the proximal end part of the cylindrical member 12.

As shown in FIG. 5, the inner circumferential surface 13a of the part from the center part to the proximal end part of the cylindrical member 13 is brought into close contact with the outer circumferential surface 23a of the outer sheath resin 23 of the flexible tube portion 9 by the swaging processing of striking the outer circumferential portion of the connecting member 11 over the whole circumference with a die 31, while moving the connecting member 11 along the axial direction.

The outer diameter of the proximal end side part 13L of the cylindrical member 13 shown by the dotted lines in FIG. 5 is reduced and also the thickness is reduced by the swaging processing as shown by the solid lines. As a result, the gap g becomes a gap g1 having a width narrower than the width of the gap in the state shown in FIG. 3.

The above-described FIG. 2 illustrates the cross section of the connecting part of the bending portion 8 and the flexible tube portion 9 after the swaging processing. The outer circumferential surface of the proximal end side part 13L of the cylindrical member 13 of the connecting member 11 is an outer circumferential surface subjected to the swaging processing. The swaging processing causes the connecting member 11 to crush the braid 22 and the outer sheath resin 23 by pressing over the length L1 of the proximal end side part 13L of the cylindrical member 13, thereby enabling the distal end thin wall portion of the flexible tube portion 9 to be firmly held over the distance L1 with a predetermined pressure in the sandwiched manner by the outer circumferential surface 12c of the proximal end side part 12L of the cylindrical member 12 and the inner circumferential surface 13a of the proximal end side part 13L of the cylindrical member 13.

Therefore, according to the present embodiment, the outer circumferential surface 12c of the proximal end side part 12L of the cylindrical member 12 and the inner circumferential surface 13a of the proximal end side part 13L of the cylindrical member 13 hold the distal end thin wall portion 9a of the flexible tube portion 9 in the sandwiching manner so as to compress the distal end thin wall portion by the swaging processing, which enables the distal end portion of the flexible tube portion 9 to be firmly fixed by the connecting member 11.

### Modified Example 1

The connecting member 11 is configured by connecting the cylindrical members 12 and 13, which are separated members, by laser welding and the like. However, the connecting member 11 may be configured by a single member.

### Modified Example 2

In the above-described embodiment, after the two cylindrical members 12 and 13 are assembled, the distal end thin wall portion 9a of the flexible tube portion 9 is inserted into the gap g of the connecting member 11. However, the distal end thin wall portion 9a of the flexible tube portion 9 is externally fitted to the proximal end part of one of the cylindrical members, and then the other of the cylindrical members may be put over the distal end thin wall portion 9a of the flexible tube portion 9.

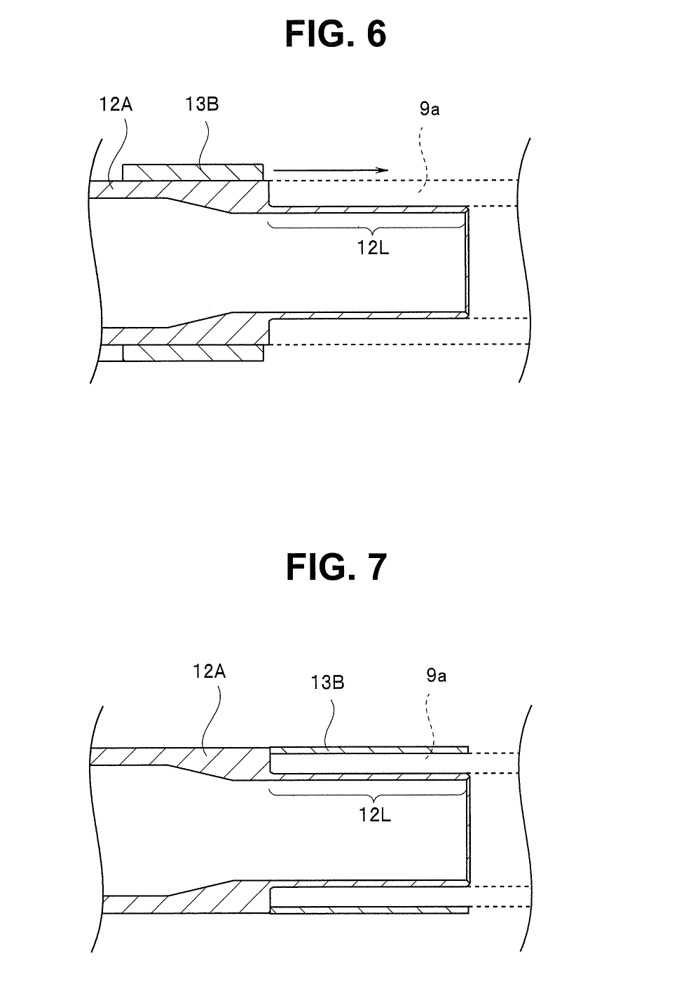

FIGS. 6 and 7 illustrate the process of externally fitting the distal end thin wall portion 9a of the flexible tube portion 9 to a proximal end side part 12L of a cylindrical member 12A, and then putting a cylindrical member 13B over the distal end thin wall portion 9a of the flexible tube portion 9, according to the modified example 2. FIG. 6 illustrates the state before the distal end thin wall portion 9a of the flexible tube portion 9 is externally fitted to a proximal end side part 12L of a cylindrical member 12A and a cylindrical member 13B is put over the distal end thin wall portion 9a of the flexible tube portion 9, according to the modified example 2. FIG. 7 illustrates a state after the swaging processing has been performed after externally fitting the distal end thin wall portion 9a of the flexible tube portion 9 to a proximal end side part 12L of a cylindrical member 12A and then putting a cylindrical member 13B over the distal end thin wall portion 9a of the flexible tube portion 9, according to the present modified example 2.

First, as shown in FIG. 6, the distal end thin wall portion 9a of the flexible tube portion 9 is externally fitted to the proximal end side part 12L of the cylindrical member 12A and fixed thereto with adhesive or the like. Then, as shown by the arrow in FIG. 6, the cylindrical member 13B is moved from the distal end side of the cylindrical member 12A to the outer circumferential portion of the distal end thin wall portion 9a of the flexible tube portion 9. Then, as shown in FIG. 7, the swaging processing is performed in the state where the cylindrical member 13B is put over the distal end this wall portion 9a of the flexible tube portion 9, which causes the distal end thin wall portion 9a of the flexible tube portion 9 to be firmly held in the sandwiched manner by the cylindrical members 12A and 13A.

### Modified Example 3

One, or two or more projection portions or an uneven portion may be formed on the inner circumferential surface 13a of the proximal end side part 13L of the cylindrical member 13 to be subjected to the swaging processing.

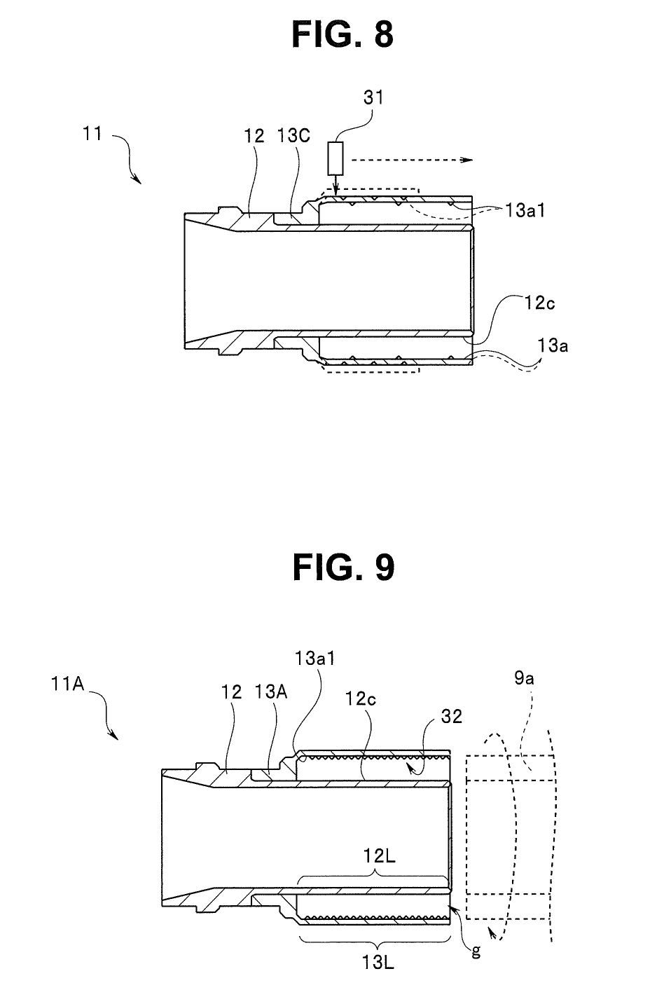

FIG. 8 is a cross-sectional view of the connecting member 11 according to the modified example 3. A cylindrical member 13C includes three projection portions 13a1 on the inner circumferential surface 13a.

When the cylindrical member 13C is pressed from the outer circumferential side by the swaging processing, the projection portions 13a1 bite the outer circumferential surface 23a of the outer sheath resin 23, and thereby the distal end portion of the flexible tube portion 9 is firmly held.

Note that the projection portions 13a1 are formed on the inner circumferential surface 13a of the cylindrical member 13 in FIG. 8. However, an uneven portion may be provided instead of the projection portions. Furthermore, the uneven portion may be what is called a granular rough surface.

### Second Embodiment

In the first embodiment, the connecting member 11 holds the distal end portion of the flexible tube 9 by the swaging processing. However, in the present embodiment, a screw portion is provided at the proximal end part of the connecting member and the connecting member holds the distal end thin wall portion 9a of the flexible tube portion 9 by the distal end thin wall portion 9a of the flexible tube portion 9 being screwed into the screw portion.

FIG. 9 is a cross-sectional view of a connecting member 11A according to the present embodiment. A female screw portion 32 is provided on the inner circumferential surface 13a of the proximal end side part 13L of the cylindrical member 13A of the connecting member 11A.

When the distal end thin wall portion 9a of the flexible tube portion 9 is screwed so as to be pushed into the gap g of the proximal end portion of the connecting member 11A while being rotated around the axis as shown by the dotted line arrow, the distal end thin wall portion 9a of the flexible tube portion 9 is fitted into the gap g while screw is threaded on the surface of the outer sheath resin 23 of the distal end thin wall portion 9a of the flexible tube portion 9.

In the state shown in FIG. 9, the distal end thin wall portion 9a of the flexible tube portion 9 is firmly held in the gap g by the cylindrical members 12 and 13A. That is, in the present embodiment, the inner circumferential surface of the proximal end side part 13L of the cylindrical member 13A has the female screw portion and the outer circumferential surface of the distal end thin wall portion 9a of the flexible tube portion 9 has a male screw portion.

Therefore, also in the present embodiment, the distal end portion of the flexible tube portion 9 can be firmly held by the connecting member 11.

As described above, according to the above-described two embodiments and the respective modified examples, it is possible to provide an endoscope in which the connection strength between the connecting member and the flexible tube portion is improved.

As a result, water and the like are not likely to enter the gap between the connecting member and the flexible tube portion and the flexible tube portion is not likely to be broken.

The present invention is not limited to the above-described embodiments, and various changes and modifications are possible within a range not changing the gist of the invention.

What is claimed is:

1. An endoscope comprising:
a bending portion;
a flexible tube portion including a tubular flexible member;
a connecting member having a cylindrical shape, the connecting member being configured to connect a proximal end portion of the bending portion and a distal end portion of the flexible tube portion; and
a holding portion configured to hold a thin wall portion of the flexible member in a sandwiching manner by a double tube portion which forms a gap formed at a proximal end portion of the connecting member and having the cylindrical shape.

2. The endoscope according to claim 1, wherein the flexible member includes a spiral tube having a shape formed by a flat plate member being wound in a spiral shape.

3. The endoscope according to claim 2, wherein the flexible member includes an outer sheath resin that covers the spiral tube.

4. The endoscope according to claim 3, wherein the flexible member includes a metal braid between the spiral tube and the outer sheath resin.

5. The endoscope according to claim 1, wherein the connecting member includes a first and second cylindrical members, and the double tube portion is formed by the first cylindrical member and the second cylindrical member that covers the first cylindrical member.

6. The endoscope according to claim 1, wherein the connecting member includes an outer circumferential surface subjected to swaging processing.

7. The endoscope according to claim 6, wherein an inner circumferential surface of the second cylindrical member includes a projection portion.

8. The endoscope according to claim 5, wherein the inner circumferential surface of the second cylindrical member includes a female screw portion, and an outer circumferential surface of the thin wall portion includes a male screw portion.
